(19) United States
(12) Patent Application Publication
(10) Pub. No.: US 2019/0021639 A1
(43) Pub. Date: Jan. 24, 2019

(54) METHEMOGLOBIN DETECTION USING PHOTOACOUSTIC IMAGING

(71) Applicant: Sunnybrook Research Institute, Toronto (CA)

(72) Inventors: Alan Moody, Toronto (CA); General Leung, Toronto (CA); Michael Kolios, Toronto (CA)

(21) Appl. No.: 16/069,608

(22) PCT Filed: Jan. 20, 2017
(86) PCT No.: PCT/CA2017/050064

§ 371 Date: Jul. 12, 2018

Related U.S. Application Data
(60) Provisional application No. 62/281,476, filed on Jan. 21, 2016.

(51) Int. Cl. A61B 5/145; A61B 5/00

(52) U.S. Cl. CPC A61B 5/14546; A61B 5/0095

(57) ABSTRACT

The present disclosure provides a method of detecting a presence of methemoglobin in a tissue-of-interest using photoacoustic techniques, the method including acquiring photoacoustic data sets at different wavelengths from the tissue-of-interest, computing a relationship between at least two of the data sets, and analyzing the relationship to determine the presence of methemoglobin in the tissue-of-interest. Methods of monitoring therapy are also disclosed.

### CROSS-REFERENCE TO RELATED APPLICATIONS

This application claims the benefit of U.S. Provisional Patent Application Ser. No. 62/281,476, filed on Jan. 21, 2016, and entitled “METHEMOGLOBIN DETECTION USING PHOTOACOUSTIC IMAGING.”

### BACKGROUND

The field of the present disclosure is systems and methods for photoacoustic imaging. More particularly, the present disclosure relates to systems and methods for detecting methemoglobin using the photoacoustic phenomena, which can indicate intraplaque hemorrhage in an atherosclerotic plaque.

Since the initial reports of a number of randomized controlled trials in the early 1990s, the degree of carotid stenosis has been the major imaging biomarker available clinically to identify those patients at increased risk of stroke. In recent years, as imaging technology has improved, it has become evident that it is now possible to identify new biomarkers predictive of future cerebrovascular events. Furthermore, these same markers have broader significance due to their association with a more generalized vascular risk phenotype. New imaging techniques integrating photoacoustics could be used to detect imaging biomarkers that would identify patients at increased vascular risk and subsequently guide clinical intervention to improve patients' risk profile.

Systemic vascular disease accounts for a large proportion of the morbidity and mortality within the adult population through heart attack, stroke, peripheral vascular and kidney diseases. Improving early detection is an opportunity to avoid these devastating conditions. Many of the drugs available have the potential to treat multiple vascular sites, and lifestyle choices (e.g., blood pressure control, cholesterol levels) can also be targeted in high risk people. The challenge is to identify which individual has occult vascular disease and is at risk of future symptomatic life-threatening disease.

Atherosclerotic plaque within the vessel wall has a complex vascular biology that, left unchecked, results in plaque progression. Beginning with endothelial dysfunction and subsequent proliferation of smooth muscle cells, plaque further responds to drivers (e.g., abnormal amounts of circulating lipid, high blood pressure) by increasing in size and complexity. As this fibroatheroma progresses, there is a proliferation and in-growth of the microvessels that feed the smooth muscle layer in the vessel (i.e., the vasa vasorum). These fragile microvessels can lead to plaque destabilization through intraplaque hemorrhage (“IPH”). Previously viewed as an incidental finding, IPH is now thought to be a significant predictor of plaque progression and destabilization. IPH can also occur through disruption of the intimal surface of the artery wall allowing ingress of blood. This may be followed by healing of the surface and potential for repetitive surface disruption.

Early histopathological studies showed that IPH was a common finding in advanced plaques. IPH is more common in endarterectomy specimens of patients who have suffered from symptomatic cerebral ischemia than asymptomatic patients. IPH also appears to be an important factor in the transformation of a low risk plaque into one that is clinically relevant. This suggests that IPH is not simply a secondary effect of plaque growth, but possibly has a causative role in plaque disruption.

Histologically detected IPH has been implicated in cholesterol crystal formation, macrophage differentiation, but perhaps mostly importantly, worse patient outcome. A large number of patients in studies that detected IPH found significant correlation with clinical outcomes. Aggregating these results, histologically found IPH had a higher prevalence in symptomatic than asymptomatic patients (p<0.0001). Most importantly, IPH detected histologically in one vascular bed appears to predict patient cardiovascular risk, suggesting that IPH in atheroma indicates a generalized increase risk in systemic cardiovascular disease. Blood out of the circulation rapidly oxidizes to methemoglobin (metHb), a highly paramagnetic state of hemoglobin which acts as an endogenous contrast agent when detected with MRI.

Methemoglobin has been associated with an environment that increases cholesterol oxidation and inflammation, both pro-atherosclerotic factors. The presence of MRI-detected metHb appears to stimulate progression of carotid plaque, identify the hemisphere of the brain that is at greater risk of embolic events, and identify plaques at higher risk of embolizing during surgery. Additionally, metHb in the carotid artery strongly predicts recurrent clinical symptoms. That is, symptomatic patients with metHb in the carotid artery are significantly more likely to have a second event than patients who do not present with this imaging biomarker.

MRI-detected metHb thus appears to be an imaging biomarker that identifies a vulnerable patient population at high risk of embolic stroke risk. Patient natural history studies and our in vitro data, suggest that metHb generates a plaque environment that predisposes a plaque to rupture through generation of reactive oxygen species and the associated inflammatory response.

Photoacoustic imaging is based on the absorption of visible light, and the conversion of the absorbed energy into an acoustic wave. The acoustic waves can then be used to reconstruct images, similar to ultrasound imaging. Scatter, which limits the depth of optical techniques, is less problematic with photoacoustic imaging as the signal localization is done by ultrasound waves that are much less affected by tissue scatter than light.

Hemoglobin in blood is one of the dominant endogenous tissue chromophores that generate photoacoustic signals. Thus, photoacoustic imaging can be used in combination with ultrasound techniques to characterize vascular structures using different wavelengths of light. The ultrasound and photoacoustic images collected are naturally co-registered. However, photoacoustic imaging has not been used to identify the presence of methemoglobin or to otherwise characterize IPH.

### SUMMARY OF THE PRESENT DISCLOSURE

The present disclosure addresses the aforementioned drawbacks by providing a method for detecting methemoglobin in a tissue-of-interest using a photoacoustic measurement system. The method includes acquiring a plurality of photoacoustic data sets at each of a plurality of different wavelengths from a tissue-of-interest using a photoacoustic measurement system. A relationship between at least two of the plurality of photoacoustic data sets is then computed and the computed relationship is analyzed to determine a presence of methemoglobin in the tissue-of-interest.

It is one aspect of the present disclosure to provide a method in which the plurality of photoacoustic data sets include at least a first photoacoustic acquired at a first wavelength and a second photoacoustic data set acquired at a second wavelength. The computed relationship can then be a ratio between the first and second photoacoustic data sets. Other photoacoustic data sets can be acquired at other wavelengths, and various combinations of ratios can be computed.

It is another aspect of the present disclosure to provide a method in which the plurality of photoacoustic data sets are acquired over a range of wavelengths. The computed relationship can then be a derivative (e.g., a first derivative, second derivative) of the signals in the photoacoustic data over the range of wavelengths.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 shows example absorption spectra for oxyhemoglobin (oHb), deoxyhemoglobin (dHb) and methemoglobin (mHb).

FIGS. 2A-2C depict a simulation of photoacoustic imaging of a carotid artery, and show an x-ray computed tomography image of a carotid artery (FIG. 2A), a schematic representation of the optical geometry for imaging the carotid artery (FIG. 2B), and example data from such a simulation (FIG. 2C).

FIGS. 3A-3E show images from an example study in which a phantom (FIG. 3A) was imaged with photoacoustic image. FIGS. 3B and 3C show photoacoustic images at wavelengths of 730 nm and 900 nm, respectively. FIG. 3D shows photoacoustic signal intensities acquired from the phantom, plotted as a function of wavelength. FIG. 3E shows a ratio map computed as a ratio between the images of FIGS. 3B and 3C.

FIG. 4 is a flowchart setting forth the steps of an example for detecting or otherwise identifying the presence of methemoglobin in a tissue-of-interest by analyzing photoacoustic signals acquired from the tissue at different wavelengths of light.

FIG. 5 is a block diagram of an example of a photoacoustic imaging system that includes a photoacoustic device.

FIGS. 6A-6D show a CT image of a subject before endarterectomy (FIG. 6A), and a T1-weighted image (FIG. 6B), photoacoustic image at 640 nm (FIG. 6C), and an HPS stained slide (FIG. 6D) of a tissue sample after the endarterectomy.

FIGS. 7A-7D show MR images (FIGS. 7A and 7B) of a subject's carotid artery, photoacoustic images of the carotid artery (FIG. 7C), and HPS stained slides (FIG. 7D) from a tissue sample from the subject.

### DETAILED DESCRIPTION

Described here are systems and methods for detecting or otherwise identifying intraplaque hemorrhage (“IPH”) using photoacoustics. As an example, photoacoustic imaging can be used in patient populations to identify asymptomatic, high risk individuals by using an imaging test that exploits the optical contrast generated by methemoglobin. In other instances, photoacoustic imaging can be used in symptomatic patient populations to identify the cause of cerebral ischemic events by using an imaging test that exploits the optical contrast generated by methemoglobin to identify carotid artery high risk atheroma.

In some embodiments, ultrasound imaging can be used to probe carotid anatomy and blood flow while using photoacoustic imaging to characterize atheroma composition. Integration of these techniques into a clinical ultrasound system would form the basis of a portable and accessible imaging modality. As one non-limiting example, a light source can be coupled to a clinical ultrasound probe used for vascular imaging. Using Doppler mode, blood flow and stenosis in the carotid artery can be characterized. Enabling the photoacoustic imaging mode will allow for characterization and detection of methemoglobin in the plaque using the methods described here.

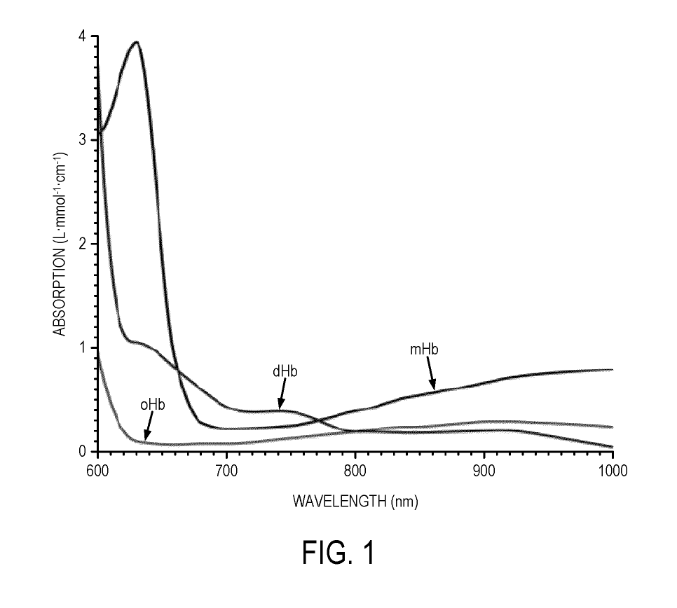

The different oxidation states of blood, oxyhemoglobin (oHb), deoxyhemoglobin (dHb), and methemoglobin (mHb), have a significantly different appearance to the naked eye. For instance, mHb typically has a black-brown appearance, which distinguishes this blood product from the more vibrant red hue that is visible from oHb and the red-purple hue that is visible from dHb. Measurement of the absorption on a spectrophotometer of these different oxidation states of blood is shown in FIG. 1, where a characteristic double hump for oHb is shown, which disappears on deoxygenation (dHb). Methemoglobin (mHb) has a distinctive peak at 630 nm not visible on the other blood products. It is contemplated, then, that detecting the change in absorption of these three blood products at different wavelengths (e.g., between 630 and 660 nm) would yield a specific signature for methemoglobin.

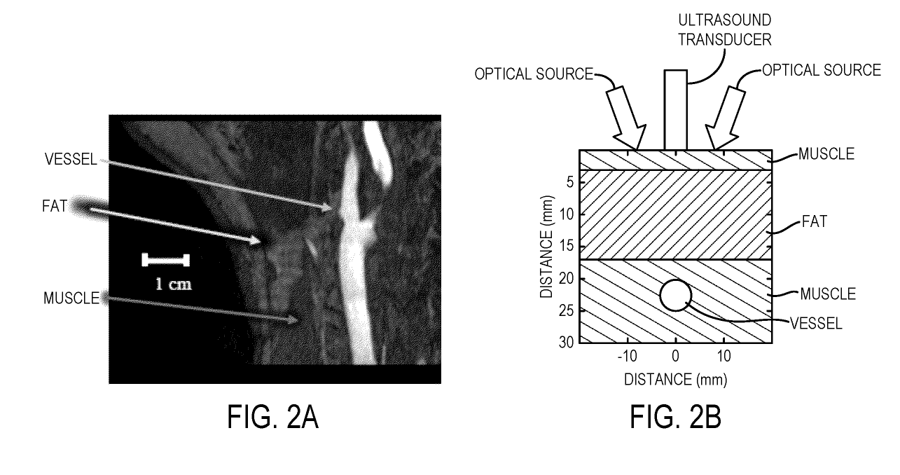

FIGS. 2A-2C depict a simulation of light and photoacoustic signal from a carotid artery to demonstrate that photoacoustic imaging is capable of delivering sufficient light to the carotid artery. For these simulations, Monte Carlo simulations were performed on an in-silico model of the carotid artery. FIG. 2A shows a parasagittal reformat of an MR angiogram of the carotid bifurcation. The average carotid bifurcations is between 2-3 cm beneath the skin surface. Although the sternocleidomastoid lies superficial to this artery, there is a pathway to the bifurcation through the subcutaneous fat, which has intermediate MR signal intensity and is located under the skin. Fat is beneficial for light propagation, owing to the low scattering and absorption. FIG. 2B shows a simulated geometry of light delivery with the two arrowheads indicating a light source, and the bar representing the ultrasound transducer. Fluence and absorption were simulated with greater than 1×10⁶ photons (using Mcxyz) and plot as a color scale (in units of dB) at both 630 nm and 1040 nm. The results of this simulation are illustrated in FIG. 2C. Highly absorbing metHb at 630 nm attenuates most of the light at the vessel surface, generating a large acoustic signal (right), eight times greater than the signal from luminal oxyHb. Depth penetration of light is somewhat improved at 1040 nm, but a lower acoustic signal, as well as metHb/oxyHb ratio is observed.

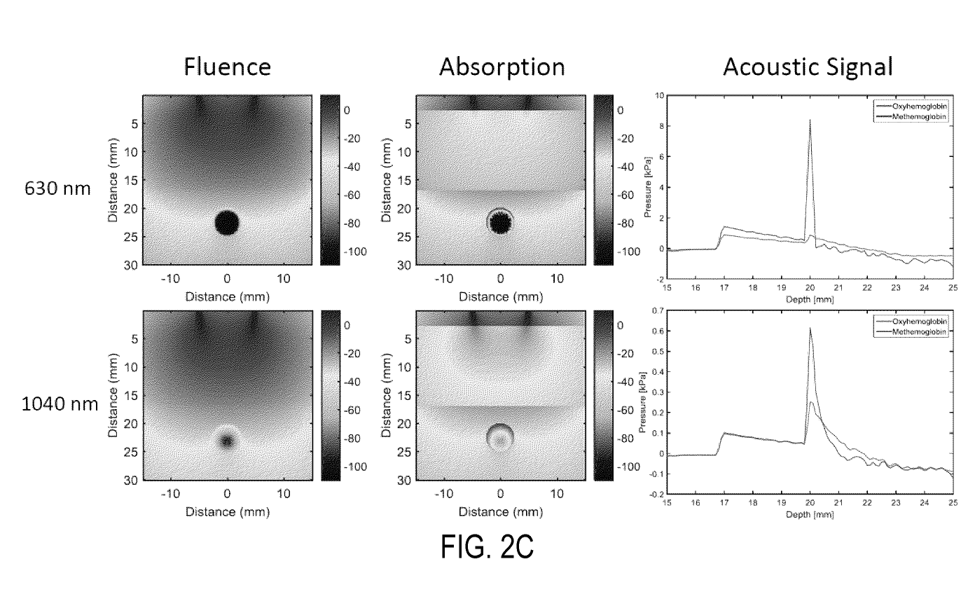

In an example study to demonstrate the ability to detect metHb from photoacoustic imaging data, a phantom with 1.5 percent agar and 5 percent intralipid emulsion (at 20 percent concentration), with two wells to contain samples, was constructed. This phantom 30 is shown in FIG. 3A. Methemoglobin and mixed venous blood (oxyHb) were inserted into the sample wells 32 and the phantom 30 was imaged on a photoacoustic imaging system, such as an iThera MSOT device, over laser wavelengths from 680 nm to 980 nm in step sizes of 10 nm. An image from 730 nm is shown in FIG. 3B and an image from 900 nm is shown in FIG. 3C. There is little discernible difference between the MetHb sample (right arrow, FIGS. 3B and 3C) and the oxyHb sample (left arrow, FIGS. 3B and 3C) in either of these images. Note also that the phantom produces no photoacoustic signal over these wavelengths.

Signal intensity in a region of interest drawn over each sample is plotted as a function of wavelength in FIG. 3D, black is the spectra from metHb, and red is the spectra from oxyHb. Distinct absorption characteristics, emission characteristics, or both, that are not visible in the photoacoustic images themselves are readily identifiable in these spectra. A ratio calculated between the image acquired at 900 nm and the image acquired at 730 nm, shown in FIG. 3E, shows a dramatic signal from the MetHb sample, an order of magnitude larger than the signal from the oxyHb sample. Thus, this ratio between photoacoustic signals at 900 nm and photoacoustic signals at 730 nm can be used as an imaging biomarker for the detection of metHb and, more particularly, IPH.

Using the ratio method described above, metHb can be uniquely identified in a scattering medium. The light propagation through tissue has been simulated using different optical wavelength combinations, as described above. Table 1 below shows some of the ranges that were examined in an experimental study. In optimizing the laser wavelengths, it is desirable to determine the ratios of absorption at various wavelengths that most favorably discriminate metHb from the other naturally occurring blood product states. It is contemplated that this will be near the metHb absorption peak of 630 nm. At this wavelength, oxyHb and deoxyHb both have absorption that is one-fourth the magnitude of the metHb absorption. Alternately, detection can be performed a higher laser length of 1040 nm, where MetHb has a two-fold higher absorption than oxyHb or deoxyHb. In some embodiments, a small number of fixed wavelength sources can be used.

TABLE 1
Optical properties of muscle, fat, oxyhemoglobin, and methemoglobin at
wavelengths 630, 700, 900, and 1040 nm.

| | | | | Scattering | |
|---|---|---|---|---|---|
| | | | | Coefficient | |
| | | Absorption | Scattering | with optical | |
| | Wavelength | Coefficient | Coefficient | clearing | Anisotropy |
| Tissue Type | (nm) | (cm) | (cm) | (cm) | parameter |
| Muscle | 630 | 0.90 | 104 | 20.8 | 0.9 |
| | 700 | 0.60 | 95.0 | 19.0 | 0.9 |
| | 900 | 0.64 | 75.0 | 15.0 | 0.9 |
| | 1040 | 0.67 | 65.2 | 13.0 | 0.9 |
| Fat | 630 | 0.01 | 157 | 31.4 | 0.9 |
| | 700 | 0.01 | 146 | 29.2 | 0.9 |
| | 900 | 0.08 | 123 | 24.6 | 0.9 |
| | 1040 | 0.05 | 112 | 22.4 | 0.9 |
| Oxyhemoglobin | 630 | 3.27 | 79.4 | 79.4 | 0.9 |
| | 700 | 1.56 | 71.4 | 71.4 | 0.9 |
| | 900 | 6.47 | 55.6 | 55.6 | 0.9 |
| | 1040 | 5.78 | 50.0 | 50.0 | 0.9 |
| Methemoglobin | 630 | 82.27 | 79.4 | 79.4 | 0.9 |
| | 700 | 2.53 | 71.4 | 71.4 | 0.9 |
| | 900 | 14.56 | 55.6 | 55.6 | 0.9 |
| | 1040 | 19.71 | 5.78 | 5.78 | 0.9 |

As mentioned above, Monte Carlo modeling was performed to examine how a small number of fixed wavelength sources will perform for different depths of the carotid artery from the skin surface (1-4 cm, see FIGS. 2A-2C). For increased sensitivity, absorption peaks in the near IR (1040 nm) or visible range (630 nm) can be used. By computing the ratio of photoacoustic signals at those wavelengths, the presence of metHb can be detected. Moreover, the absorption of blood in the near IR is less than at 600 nm, allowing adequate penetration of the light.

In some embodiments, a reflection geometry approach is implemented, in which the laser and ultrasound array are located on the same side of the tissue being imaged. Different combinations of cross laser beam geometries can be implemented, with the laser delivered through fiber optic bundles including two strips along both sides of the acoustic aperture at angles between 10-35 degrees relative to the imaging plane (in steps of five degrees).

In some embodiments, if not enough light is able to penetrate the tissue to generate a photoacoustic signal, optical clearing techniques can be implemented. In these techniques, agents are used to penetrate into tissues to replace water, thereby reducing the variations of the refractive index between scatterers. Use of these techniques have reported increases in the photoacoustic signal SNR by a factor of 1.736. Further, owing to the relatively supple structures overlying the carotid artery, a small amount of compression can bring the focus of the transducer closer to the imaging structure. Another approach for imaging the carotid artery would be to use a method in which an optical probe is placed in the pharynx, allowing the carotid artery to be illuminated from within the body, while the ultrasound detection is still positioned external to the subject.

The known problems of missing horizontal boundaries and reduced horizontal resolution, an inherent feature of any linear array geometry in photoacoustic imaging, are not critical for the methods described here because anatomical information can be provided by B-mode ultrasound imaging, Doppler ultrasound imaging, or both, and because photoacoustic imaging is used to detect the presence of IPH, not to provide high resolution spatial maps of its distribution.

Conventional ultrasound instruments use 7-12 MHz ultrasound to image the carotid artery. However, as light generates the photoacoustic signal, the ultrasound wave only has half the distance to travel in photoacoustic imaging applications. Therefore, frequencies up to 20 MHz can be used for photoacoustic detection. Even though the photoacoustic detection of metHb does not require high spatial resolution (because the ultrasound B-mode/Doppler can be used to guide imaging), it may provide gains in sensitivity for the detection of small pockets of metHb.

It is recognized that different values of optical absorption and scattering as a function of wavelength may result in different optical illumination patterns. These different optical illumination patterns can be used to make appropriate corrections for the change in the anticipated photoacoustic signal intensity. In clinical scenarios, because the general location of plaque is known from ultrasound imaging, reasonable estimates can be made of the effect of the intervening tissue on the optical illumination patterns. Knowledge of the general location of the plaque can also help in specialized beamforming techniques.

If the linear array transducer geometry does not provide adequate SNR despite optimized optical delivery, software or hardware approaches can be used to increase the SNR. As one example of a software approach, an aperture domain filtered spatial compounding image reconstruction method can be used to enhance image CNR and target detectability. Photoacoustic images generally suffer from artifacts due to signals produced from off-axis objects (e.g., the skin can produce strong signals). The different spatial orientation of the signals from the on-axis and off-axis objects can be characterized as different spatial frequencies after applying a 2D Fourier transform over aperture directions of the receive elements of the linear array. Filtering this with a 2D aperture domain filter has been shown to be effective in removing the off-axis signal in ultrasound.

As one example of a hardware approach, instead of a linear array, a curved array can be used. As another example of a hardware approach, an annular array can be used. Nevertheless, it is contemplated that a linear array will provide sufficient sensitivity to detect metHb in the human carotid artery.

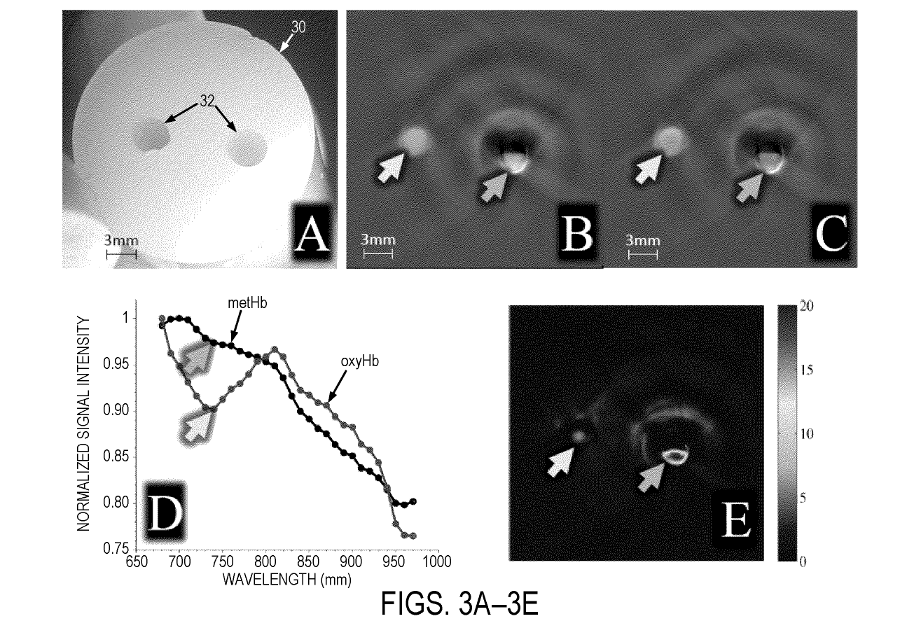

Referring now to FIG. 4, a flowchart is illustrated as setting forth the steps of an example of a method for detecting or otherwise identifying the presence of methemoglobin by analyzing photoacoustic signals. The method begins with the acquisition of photoacoustic data from a tissue-of-interest at a first wavelength, as indicated at step 402. The tissue-of-interest may be an artery, vein, or other bodily tissue. Photoacoustic data are acquired using an appropriate photoacoustic measurement system, such as a photoacoustic spectroscopy or photoacoustic imaging system. Photoacoustic data are then acquired from the tissue-of-interest at a second wavelength, as indicated at step 404. As one example, the first wavelength can be at or around 630 nm and the second wavelength can be at or around 1040 nm.

In some embodiments, more than two photoacoustic data sets can also be acquired by imaging the tissue-of-interest at more than two wavelengths. Thus, in general, a plurality of photoacoustic data sets can be acquired at a plurality of different wavelengths. The wavelengths at which photoacoustic data are acquired can be selected to correspond to optical characteristics of chromophores other than methemoglobin. For instance, the wavelengths can be selected to correspond to optical characteristics of other chromophores in the tissue-of-interest (e.g., oxyhemoglobin, deoxyhemoglobin, melanin).

A relationship between the photoacoustic signals, representing the absorption spectra of the tissue-of-interest, is then computed, as indicated at step 406, and described above. As one example, the relationship can be a ratio between two photoacoustic data sets, such as a ratio between photoacoustic data acquired at a first wavelength (e.g., 630 nm) and photoacoustic data acquired at a second wavelength (e.g., 1040 nm). Ratios can also be computed between different chromophores (e.g., oxyhemoglobin, deoxyhemoglobin, melanin) by acquiring photoacoustic data at wavelengths associated with those chromophores and computing ratios using on those other data sets. In some embodiments, this ratio is computed on a pixel-by-pixel basis by computing the ratio of photoacoustic images at the first and second wavelengths generated from the respective photoacoustic data. Thus, a ratio map can be generated in this manner.

As another example, the relationship can be a slope or second derivative of the absorption curve over a range of wavelengths (e.g., 630-700 nm). Thus, in these instances it may be preferable to acquire photoacoustic data from more than two wavelengths over the range of wavelengths (e.g., by sampling the range of wavelengths in 10 nm increments). In some embodiments, the slope or second derivative can be computed on a pixel-by-pixel basis, thereby generating a slope or second derivative map.

The computed relationships are then analyzed to detect the presence of metHb, as indicated at step 408. As one example, the analysis can include analyzing a ratio, slope, second derivative, or other relationship map to identify regions of image intensity values (e.g., ratio values, signal change values) above a particular threshold value indicative of the presence of metHb.

By analyzing these spectral characteristics from multiple wavelengths to detect metHb, a biomarker indicative of different pathological or physiological states in the tissue-of-interest can be generated. As one example, the tissue-of-interest can be an artery and the detection of metHb can be a biomarker indicating IPH. As another example, the tissue-of-interest can be a vein and the detection of metHb can be a biomarker indicating deep vein thrombosis (“DVT”). Accordingly, the presence of metHb can be detected or otherwise identified from photoacoustic imaging data and related to pathological or physiological states of the tissue-of-interest, such as whether IPH or DVT may be present. These biomarkers can also be monitored over time during a treatment regiment to monitor or otherwise asses the efficacy of that particular treatment.

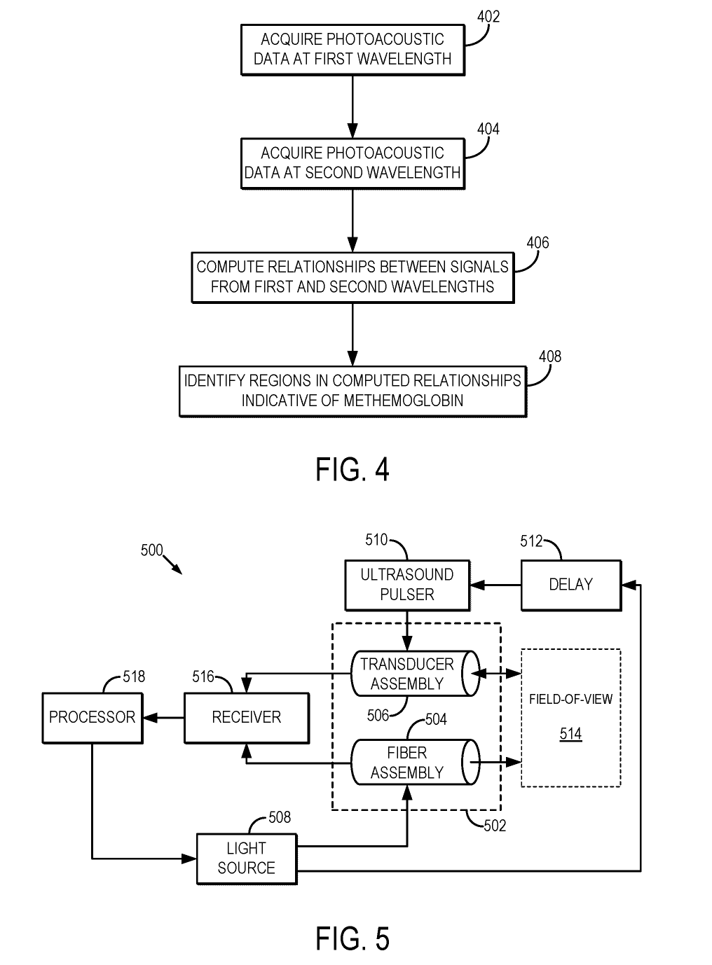

Referring now to FIG. 5, a block diagram of an example photoacoustic imaging system 500 that incorporates a photoacoustic imaging device 502 is illustrated. The photoacoustic imaging device 502 generally includes a fiber assembly 504 and a transducer assembly 506, which may be coupled together. For instance, the fiber assembly 504 and transducer assembly 506 may be coupled via a common outer sheath that holds the fiber assembly 504 and transducer assembly 506 in spaced arrangement.

The fiber assembly 504 includes at least one optical fiber. A light source 508 is optically coupled to the fiber assembly 504 and delivers light to the distal end of the fiber assembly 504 to irradiate the object being imaged. In some embodiments, the light source 508 is a laser source, which may be a continuous wave laser source or a pulse wave laser source. In some embodiments, the light source 508 may include multiple laser systems or diodes, thereby providing different optical wavelengths, that are fed into one or more optical fibers. The selection of an appropriate excitation wavelength for the light source 508 is based on the absorption characteristics of the imaging target. Because the average optical penetration depth for intravascular tissue is on the order of several to tens of millimeters, the 400-2100 nm wavelength spectral range is suitable for intravascular photoacoustic applications. Thus, in some embodiments, the light source 508 may be an Nd:YAG (neodymium-doped yttrium aluminum garnet) laser that operates at 1064 nm wavelength in a continuous mode.

The transducer assembly 506 generally includes an photoacoustic transducer for receiving photoacoustic signals generated by an illumination field, such as an illumination field generated by pulsed or continuous wave laser light. In some configurations, the photoacoustic transducer can also be operated to generate ultrasound energy and to receive pulse-echo ultrasound emissions. In this configuration, the photoacoustic transducer can be operated in a receive-only mode for photoacoustic imaging and, when the illumination field is not being generated, the photoacoustic transducer can also be operated in an ultrasound imaging mode to obtain ultrasound images. In some configurations, the photoacoustic transducer may include multiple transducer elements, some of which may be dedicated solely for receiving photoacoustic signals while others may be dedicated solely to generating and receiving pulse-echo ultrasound signals.

In some other configurations, the transducer assembly 506 may include at least two transducers: a dedicated photoacoustic transducer and a dedicated ultrasound transducer for generating and receiving pulse-echo ultrasound signals. In this dual-transducer configuration, both photoacoustic and ultrasound images can be obtained. With the dual-transducer configuration, photoacoustic and ultrasound images can be obtained simultaneously and, even when not obtained simultaneously, are innately co-registered given the spatial relationship between the photoacoustic transducer and the ultrasound transducer.

Irradiation with the light source 508 is performed at a given point for a finite amount of time with an optical excitation waveform. In frequency-domain photoacoustic applications, in which a continuous wave laser is used, the optical excitation waveform may be amplitude modulated with frequency sweeping, such as a chirp or pulse train. The irradiation produced by this type of optical excitation results in a frequency-domain photoacoustic modulated signal being produced in the region illuminated by the photoacoustic imaging device 502. The chirp can include a multitude of different excitation waveforms including linear, non-linear, and Gaussian tampered frequency swept chirps.

When used to obtain ultrasound images, operation of the transducer assembly 506 may be controlled by an ultrasound pulser 510, which provides ultrasound excitation waveforms to the transducer assembly 506. In single-transducer configurations in which the photoacoustic transducer is used to both receive photoacoustic signals and to generate and receive pulse-echo ultrasound signals, a delay 512 between the light source 508 and the ultrasound pulser 510 provides a trigger signal that directs the ultrasound pulser 510 to operate the photoacoustic transducer at a delay with respect to the irradiation of the field-of-view 514. The timing provided by the delay 512 enables the detection of photoacoustic signals by the photoacoustic transducer in the transducer assembly 506 when the field-of-view 514 is being illuminated, but also the generation and detection of pulse-echo ultrasound signals when the field-of-view 514 is not being illuminated.

Signals received by the transducer assembly 506 are communicated to a receiver 516, which generally includes a pre-amplifier, but may also include one or more filters, such as bandpass filters for signal conditioning. The received signals are then communicated to a processor 518 for analysis.

Thus, the generated photoacoustic signals are detected by the photoacoustic transducer in the transducer assembly 506, communicated to the receiver 516, and then communicated to the processor 518 for processing and/or image generation. As one example, the photoacoustic signals can be processed in accordance with the methods described above to monitor the physical changes in a tissue following the administration of a treatment, such as a radiation treatment or a chemotherapy treatment. Similarly, pulse-echo ultrasound signals received by either the photoacoustic transducer or a dedicated ultrasound transducer in the transducer assembly 506 can also be communicated to the receiver 516 and then communicated to the processor 518 for processing and/or image generation.

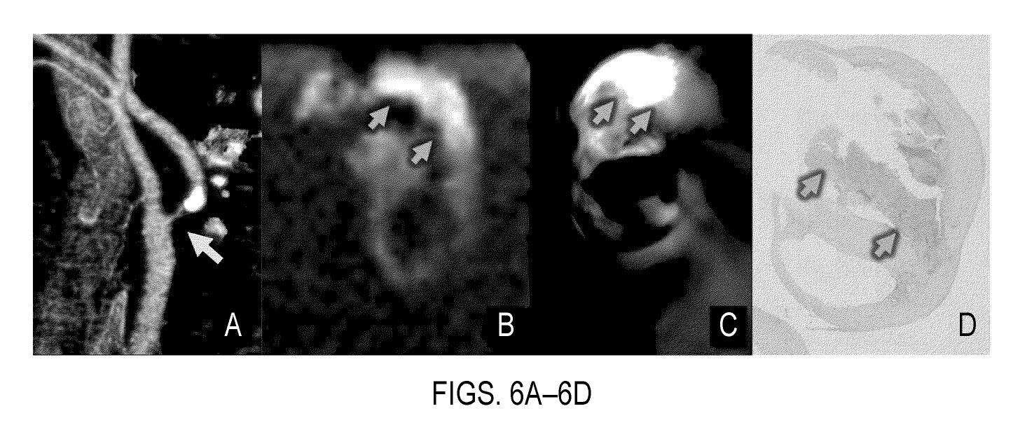

FIG. 6A shows a parasagittal reformat of a CT angiogram of the carotid bifurcation in a patient scheduled for endarterectomy. A very tight stenosis is visualized (arrowhead), with a significant calcification more distal in the external carotid artery. After endarterectomy, high resolution (0.2 mm isotropic), T1 weighted MRI of the tissue specimen is shown in FIG. 6B. A signal hyperintensity, associated with intraplaque hemorrhage (IPH), is visualized on the anterior aspect of this plaque specimen (arrowheads). Calcification of the sample generates signal voids within the plaque, (between the arrowheads). FIG. 6C shows a single slice of a photoacoustic image of this same specimen imaged at 640 nm. Again, a clear signal hyperintensity is seen in the area associated with IPH (arrowheads). Unlike the MR image, however, no distortion of the image is generated from the calcification using the photoacoustic imaging system. FIG. 6D shows an HPS stained slide of this endaraterectomy specimen, confirming plaque hemorrhage in the same location as detected by both non-invasive imaging modalities. This histology is slightly distorted owing to the decalcification process.

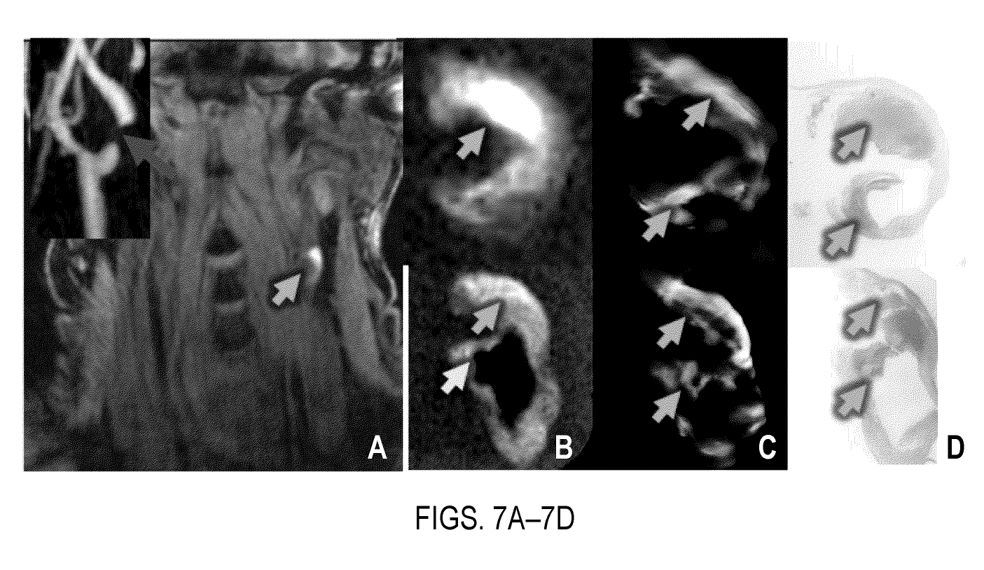

FIGS. 7A-7D illustrate an example of light and photoacoustic signal from a carotid artery in a patient with an MRI positive IPH plaque at the bifurcation of the left carotid artery (arrowhead), shown in FIG. 7A. The inset on FIG. 7A shows the MR angiogram demonstrating a near total occlusion (arrowhead). After carotid endarterectomy, this specimen was re-imaged again with MRI (FIG. 7B), and with photoacoustic imaging (FIG. 7C) and stained with HPS (FIG. 7D). The MR images show signal hyperintensity corresponding to histologically proven intraplaque hemorrhage. The photoacoustic images can be color coded to indicate areas where spectral reconstruction matches the spectra of MetHb. Photoacoustic imaging detects an area of hemorrhage (lowest arrowhead, FIGS. 7C and 7D) that corresponds with histological IPH and is missed by MR (lowest arrowhead, FIG. 7B).

The present disclosure has described one or more preferred embodiments, and it should be appreciated that many equivalents, alternatives, variations, and modifications, aside from those expressly stated, are possible and within the scope of the invention.

1. A method for detecting methemoglobin in a tissue-of-interest using a photoacoustic measurement system, the steps of the method comprising:
(a) acquiring a plurality of photoacoustic data sets at each of a plurality of different wavelengths from a tissue-of-interest using a photoacoustic measurement system;
(b) computing with a processor, a relationship between at least two of the plurality of photoacoustic data sets; and
(c) analyzing the computed relationship with the processor to determine a presence of methemoglobin in the tissue-of-interest.

2. The method as recited in claim 1, wherein step (a) includes acquiring first photoacoustic data with the photoacoustic measurement system at a first wavelength from the tissue-of-interest and acquiring second photoacoustic data with the photoacoustic measurement system at a second wavelength from the tissue-of-interest.

3. The method as recited in claim 2, wherein the first wavelength is at or near 630 nanometers.

4. The method as recited in claim 2, wherein the second wavelength is at or near 1040 nanometers.

5. The method as recited in claim 2, wherein step (b) includes computing the relationship between at least two of the plurality of photoacoustic data sets by computing a ratio between the first photoacoustic data and the second photoacoustic data.

6. The method as recited in claim 5, wherein step (b) includes producing a first photoacoustic image from the first photoacoustic data and a second photoacoustic image from the second photoacoustic data, and computing the ratio includes generating a ratio map by computing a ratio between the first and second photoacoustic images.

7. The method as recited in claim 6, wherein step (c) includes analyzing the ratio map to identify regions in the ratio map having image intensity values above a threshold value.

8. The method as recited in claim 7, wherein the first wavelength is at or near 630 nanometers, the second wavelength is at or near 1040 nanometers, and the threshold value is about 10.

9. The method as recited in claim 1, wherein:
step (a) includes acquiring first photoacoustic data with the photoacoustic measurement system at a first wavelength from the tissue-of-interest, acquiring second photoacoustic data with the photoacoustic measurement system at a second wavelength from the tissue-of-interest, and acquiring third photoacoustic data with the photoacoustic measurement system at a third wavelength from the tissue-of-interest; and
step (b) includes computing the relationship between at least two of the plurality of photoacoustic data sets by computing a ratio between at least two of the first, second, and third photoacoustic data sets.

10. The method as recited in claim 1, wherein the plurality of different wavelengths corresponds to a range of wavelengths and step (b) includes computing the relationship by computing a derivative of photoacoustic signals in the plurality of photoacoustic data sets over the range of wavelengths.

11. The method as recited in claim 10, wherein the derivative is at least one of a first derivative or a second derivative.

12. The method as recited in claim 1, wherein the tissue-of-interest includes tissue in an artery and further comprising generating a report that provides a biomarker indicating intraplaque hemorrhage in the artery based on the determined presence of methemoglobin.

13. The method as recited in claim 12, wherein the artery is at least one of a carotid artery or a coronary artery.

14. The method as recited in claim 1, wherein the tissue-of-interest includes tissue in a vein and further comprising generating a report that provides a biomarker indicating deep vein thrombosis in the vein based on the determined presence of methemoglobin.

15. A method for detecting methemoglobin in a tissue-of-interest using a photoacoustic measurement system, the steps of the method comprising:
(a) acquiring first photoacoustic data at a first wavelength from a tissue-of-interest using a photoacoustic measurement system;
(b) acquiring second photoacoustic data at a second wavelength from the tissue-of-interest using the photoacoustic measurement system;
(c) computing with a processor, a quantitative relationship between the first photoacoustic data and the second photoacoustic data; and
(d) analyzing the computed quantitative relationship with the processor to determine a presence of methemoglobin in the tissue-of-interest.

16. The method as recited in claim 15, wherein the first wavelength is associated with an optical characteristic of a first chromophore and the second wavelength is associated with an optical characteristic of a second chromophore.

17. The method as recited in claim 15, wherein the first wavelength is different from the second wavelength, and the first wavelength and the second wavelength are selected from the group consisting of 630 nanometers, 730 nanometers, 900 nanometers, and 1040 nanometers.

18. The method as recited in claim 15, wherein computing the quantitative relationship in step (c) includes computing a ratio between the first photoacoustic data and the second photoacoustic data.

19. The method as recited in claim 15, further comprising:
acquiring with the photoacoustic measurement system, additional photoacoustic data from the tissue-of-interest at one or more different wavelengths in a range of wavelengths between the first wavelength and the second wavelength; and
wherein computing the quantitative relationship in step (c) includes computing a derivative of an absorption curve spanning the range of wavelengths between the first wavelength and the second wavelength and defined by the first photoacoustic data, the second photoacoustic data, and the additional photoacoustic data.

20. The method as recited in claim 19, wherein the derivative is one of a first derivative or a second derivative.

21. The method as recited in claim 19, wherein the first wavelength is 630 nanometers and the second wavelength is 700 nanometers.
